(19) United States
(12) Patent Application Publication
(10) Pub. No.: US 2026/0301944 A1
(43) Pub. Date: Oct. 1, 2026

(54) CLASSIFIER AND TRAINING SYSTEM FOR EARLY IDENTIFICATION AND SEVERITY PREDICTION OF PLACENTA ACCRETA SPECTRUM

(71) Applicant: CHANGZHOU MATERNAL AND CHILD HEALTH CARE HOSPITAL, Jiangsu (CN)

(72) Inventors: Zhonghua SHI, Jiangsu (CN); Runrun HAO, Jiangsu (CN); Bin ZHANG, Jiangsu (CN); Xueqi BAI, Jiangsu (CN); Shanshan WANG, Jiangsu (CN); Shiman HU, Jiangsu (CN); Sutong KAN, Jiangsu (CN); Haoyan SHI, Jiangsu (CN)

(73) Assignee: CHANGZHOU MATERNAL AND CHILD HEALTH CARE HOSPITAL, Jiangsu (CN)

(21) Appl. No.: 19/344,564

(22) Filed: Sep. 30, 2025

(30) Foreign Application Priority Data
Sep. 30, 2024 (CN) 202411383044.7

(51) Int. Cl. G16H 50/20

(52) U.S. Cl. CPC G16H 50/20

(57) ABSTRACT

A classifier and a training system for early identification and severity prediction of placenta accreta spectrum. This system implements normalization and discretization based on analysis with genome-wide maternal plasma cell-free DNA promoter coverages, and identifies an optimal target combination with 15 genes including MAPK10, MIR3169, MIR12133, and the like, demonstrating potential for preparation of a diagnostic kit. Based on a machine learning algorithm, the constructed classifier exhibits high sensitivity and specificity in early prediction of PAS and severity thereof, and areas under the receiver operating characteristic curve (AUC) all exceed 0.85, effectively implementing early risk assessment for high-risk pregnant women and identification of severe PAS of pregnant women, to reduce critical and life-threatening obstetric conditions. This classifier clinically provides a non-invasive predictive tool, holding significant clinical application value and medical significance.

### CROSS-REFERENCE TO RELATED APPLICATIONS

This application claims the priority benefit of China application serial no. 202411383044.7, filed on Sep. 30, 2024. The entirety of the above-mentioned patent application is hereby incorporated by reference herein and made a part of this specification.

### BACKGROUND

### Technical Field

The present invention relates to the field of model prediction, and in particular, to a classifier and a training system for early identification and severity prediction of placenta accreta spectrum.

### Background

Placenta accreta spectrum (PAS) includes placenta accreta (PA), placenta increta (PI), and placenta percreta (PP), especially PI and PP are leading causes of critical and life-threatening obstetric conditions and maternal death. Prenatal diagnosis of severe PAS primarily relies on imageological examination based on existing methods, with many limitations: (1) B-mode ultrasonography excels in assessment of PAS blood but is limited in evaluation of attachment depth and extent; (2) magnetic resonance imaging (MRI) offers no advantage in assessment of PAS blood, is costly, not universally available in specialized hospitals, and requires highly skilled radiologists; (3) a diagnostic window of imageological examination is in mid-to-late pregnancy, facing a dilemma for definitive diagnosis; and (4) in 36% of patients, preoperative imaging findings do not correlate with intraoperative findings. Therefore, early identification of severe PAS and advancement of the diagnostic window and accurate prediction of PAS to early-to-mid pregnancy hold significant clinical importance for high-risk referrals of PI and PP pregnant women, multidisciplinary consultations, and reduction of risks in pregnant or lying-in women and perinatal infants.

Non-invasive prenatal testing (NIPT) based on low-coverage whole-genome sequencing is clinically common for prenatal screening. In recent years, NIPT has shown significant value not only in screening for fetal chromosomal abnormalities, but also in early prediction of pregnancy complications, such as fetal growth restriction, macrosomia, and preeclampsia, but has not been applied to early prediction of PAS. Nucleosome coverage footprints extracted from cell-free DNA (cfDNA) at promoter regions in plasma during early pregnancy can reflect gene expression patterns of tissue of origin such as placenta and decidua, demonstrating extremely high predictive potential for placenta-derived diseases, particularly PAS, and its severity.

### SUMMARY

To address practical needs and drawbacks in the related art, the present invention provides a classifier and a training system for early identification and severity prediction of placenta accreta spectrum, to resolve current lack of methods for accurately predicting occurrence and severity of PAS during early-to-mid pregnancy.

### Technical Solutions

To achieve the foregoing objective, the present invention provides the following technical solutions:

According to a first aspect, the present invention provides a classifier for early identification of a PAS risk and severity in high-risk pregnant women based on plasma cfDNA promoter coverages, where a target gene combination includes MAPK10, MIR3169, MIR12133, ZDHHC6, LOC644634, LOC728739, KRT33A, C1GALT1C1, MIR1263, RBFOX2, CEP192, LINC02330, LPCAT3, OR7E2P, and OR4N2.

In the present invention, genome-wide cfDNA promoter coverage profiles are discovered in plasma of pregnant women with occurrence of PAS during early-to-mid pregnancy based on NIPT data, an optimal gene combination and an optimal cutoff value of each gene are obtained based on machine learning strategies (see a second aspect) to train a classifier, and an area under the curve (AUC) of the receiver operating characteristic curve (ROC) is predicted in an independent validation dataset, which reaches 0.85 or more, demonstrating good potential as a screening means for PAS in high-risk pregnant women.

A method for assessing a placenta accreta spectrum risk and severity in high-risk pregnant women includes the following steps:

Data collection and preprocessing: collect low-coverage whole-genome sequencing data of high-risk pregnant women undergoing NIPT, and perform necessary preprocessing, to ensure data quality.

Promoter region identification and coverage extraction: align NIPT data to human reference genome hg19 by using software bwa-mem, SAMtools, and BEDtools and a sequence alignment algorithm, and determine promoter regions pTSS from −1000 bp to +1000 bp around transcription start sites (TSS) of 15 genes: MAPK10, MIR3169, MIR12133, ZDHHC6, LOC644634, LOC728739, KRT33A, C1GALT1C1, MIR1263, RBFOX2, CEP192, LINC02330, LPCAT3, OR7E2P, and OR4N2, to obtain original read coverages of the 15 genes at the pTSS regions.

Feature factor normalization: normalize the original read coverages of the 15 genes at the pTSS regions by a TPM-like method, to obtain normalized pTSS coverages (NPC) of the 15 genes.

$${NPC}_{i} = \frac{{q}_{i} / {l}_{i}}{{\sum }_{j} ( {q}_{j} / {l}_{j} )} * {10}^{6} = \frac{{q}_{i}}{{\sum }_{j} {q}_{j}} * {10}^{6}$$

wherein, NPCᵢ represents a normalized pTSS coverage of a gene i, qᵢ represents an original read coverage at the pTSS region, lᵢ represents a transcript length (all being 2000), and Σⱼ(qⱼ/lⱼ) represents a sum of pTSS read coverages of all genes normalized based on the transcript length in one sample.

Feature factor discretization: compare the NPC values of the 15 genes respectively to optimal cutoff values (obtained in the second aspect) of all the genes. A case in which a feature factor NPC is greater than a corresponding optimal cutoff value is set to 1. A case in which a feature factor NPC is not greater than a corresponding optimal cutoff value is set to 0.

Risk assessment: input the discretized NPC values of the 15 genes of pregnant women to the classifier, to calculate a severe or mild PAS risk.

This method is applicable to singleton pregnancy with at least one of the following high-risk factors of PAS: (1) history of uterine surgery, such as cesarean section, myomectomy, and uterine septum resection; (2) history of intrauterine procedures, such as hysteroscopic surgery and curettage; and (3) pregnancy achieved via in vitro fertilization-embryo transfer (IVF-ET).

This process provides a non-invasive assessment means through the extraction of the pTSS coverage based on the NIPT data. This indicates that a particular combination and an expression pattern of the 15 genes demonstrate significant potential for preparation of a diagnostic kit. During development of the diagnostic kit, the expression of these genes is detected by using an accurate molecular diagnosis technique. A detection method may be designed with high sensitivity and high specificity, to implement accurate early identification and risk prediction for PAS high-risk pregnant women, with the potential to become a routine clinical detection item, serving a broader patient population.

According to a second aspect, this patent provides a three-class classifier training system for stratified prediction of PAS based on plasma cfDNA promoter sequencing during early-to-mid pregnancy.

The three-class classifier training system for stratified prediction of PAS based on plasma cfDNA promoter sequencing during early-to-mid pregnancy includes the following modules:

A dataset division module is configured to extract pre-collected medical data of PAS high-risk pregnant women at NIPT on different sequencing platforms, match pregnant women with occurrence of PAS and pregnant women without occurrence of PAS based on maternal age, gestational age at NIPT, fetal sex, and distribution of high-risk factors, randomly divide samples from a primary platform into a training dataset and an internal validation dataset, and use samples from the remaining platform as an independent external validation dataset. According to the latest FIGO diagnostic standard, a panel of obstetric specialists classifies PAS pregnant women into PP, PI, and PA based on surgical records. Pregnant women with occurrence of PP or PI are defined as a severe group, and pregnant women with occurrence of PA are defined as a mild group.

An impact factor extraction module is configured to perform annotation on pre-collected NIPT data of the PAS high-risk pregnant women with cfDNA promoter nucleosome coverage profiles and perform feature extraction.

The NIPT is low-depth high-throughput sequencing on cell-free DNA in maternal peripheral blood. The NIPT data is aligned to human reference genome hg19 by using an accurate sequence alignment algorithm and using software bwa-mem, SAMtools, and BEDtools, PCR duplicates are removed, a region from −1000 bp to +1000 bp around a transcription start site (TSS) is determined as a promoter region pTSS, and an original read coverage at the pTSS region is calculated.

TPM-like calculation is performed to obtain a normalized pTSS coverage (NPC) of each gene, to reduce an impact of a sequencing depth on data extraction and analysis.

$${NPC}_{i} = \frac{{q}_{i} / {l}_{i}}{{\sum }_{j} ( {q}_{j} / {l}_{j} )} * {10}^{6} = \frac{{q}_{i}}{{\sum }_{j} {q}_{j}} * {10}^{6}$$

wherein, NPCⱼ represents a normalized pTSS coverage of a gene i, qᵢ represents an original read coverage at the pTSS region, lᵢ represents a transcript length (all being 2000), and Σⱼ(qⱼ/lⱼ) represents a sum of pTSS read coverages of all genes normalized based on the transcript length in one sample.

NPC values corresponding to all genes are separately input to the system as impact factors.

A feature selection module is configured to perform screening on the impact factors by using the following strategy:

High-risk pregnant women in a severe PAS group and high-risk pregnant women without occurrence of PAS are matched at 1:1 and high-risk pregnant women in a mild PAS group and high-risk pregnant women without occurrence of PAS are matched at 1:1 in the training dataset by propensity scoring, and pairwise differential analyses are performed on the three matched groups. The impact factors in each two groups are subjected to three differential analysis methods: DESeq2, limma-voom, and a rank-sum test, to obtain impact factors with a p value<0.05 as the feature factors in the two groups. Intersection analysis is performed on three groups of feature factors obtained respectively through three times of screening, and feature factors obtained through all three times of screening or through all at least two times of screening are selected as optimal feature factors and included in a next module.

A feature factor discretization module is configured to implement a discretization strategy to enhance universality and clinical utility of the classifier for different sequencing platforms. One group with an average NPC value of a gene significantly higher or lower than the other two groups in the three groups is used as a specificity group of the gene. The optimal cutoff value is defined as a NPC value with a maximum Youden index between the specificity group and the other two groups (that is, a maximum sum of sensitivity and specificity). A case in which a feature factor NPC is greater than a corresponding optimal cutoff value is set to 1; and a case in which a feature factor NPC is not greater than a corresponding optimal cutoff value is set to 0.

A model acquisition module is configured to input the feature factors obtained through screening to a machine learning feature selection process, for example, recursive feature elimination (RFE), and gradually construct a PAS prediction classifier by using machine learning such as SVM-gaussian kernel function (radial basis function, RBF).

Pregnant women in the severe PAS group that are set to 2, pregnant women in the mild PAS group that are set to 1, and non-PAS high-risk pregnant women set to 0 are input to the system for training two binary classifiers 0-(1+2) and 1-2. To increase sensitivity of the classifiers, the class weight parameter is set to {0:0.1, 1:0.3}. An optimal feature factor combination is separately extracted, and a classifier and an assessment result thereof are output.

Optimal feature factor combinations of the two binary classifiers 0-(1+2) and 1-2 are combined and then input to the machine learning feature selection process, for example, recursive feature elimination (RFE), and a three-class 0-1-2 PAS prediction classifier is constructed by using a plurality of machine learning algorithms such as SVM-gaussian kernel function (radial basis function, RBF). An optimal feature factor combination is extracted, and a classifier and an assessment result thereof are output, including average performance of the classifier and identification results of PAS of all types.

### Beneficial Effects of the Present Invention

PAS is a collective term for a group of diseases characterized by abnormal placental adhesion or invasion into myometrium. The placenta of a fetus delivered from a PAS patient fails to detach normally, causing massive blood loss on a placental separation surface. This is a leading cause of critical and life-threatening obstetric conditions such as emergency hysterectomy, multiple organ failure, disseminated intravascular coagulation, and shock, and even perinatal mortality. In recent years, the incidence of PAS has risen annually due to increased cesarean sections, and development in intrauterine procedures and assisted reproductive technologies. Statistics indicate that PAS occurs in one of every 300 to 400 cases of pregnancy. Although PAS cases diagnosed prenatally using an existing clinical method usually involve severe invasion into the myometrium, they exhibit lower rates of emergency cesarean section, blood loss, and blood transfusions than PAS cases undiagnosed prenatally. Therefore, prenatal identification and perinatal management of PAS are critically important.

This study develops a classifier based on NPC of 15 genes, which can effectively implement stratified prediction of a PAS risk in high-risk pregnant women based on non-invasive blood testing during early-to-mid pregnancy, demonstrating significant clinical application value. The introduction of this innovative method holds promise for direct application in clinical practice, offering a novel and safe prediction means for early prevention, clinical treatment, and health management for PAS, and demonstrating great clinical significance for identifying pregnancy risks, promoting maternal and infant health, and implementing source prevention and control for critical and life-threatening obstetric conditions.

### BRIEF DESCRIPTION OF THE DRAWINGS

FIG. 1 is a Venn diagram of differential gene screening in Example 1

FIG. 2 is a schematic diagram of performance of a 23-gene SVM-RBF kernel 0-(1+2) classifier obtained by inputting NPC values of 211 genes in Example 1

FIG. 3 is a schematic diagram of performance of a 15-gene SVM-RBF kernel 1-2 classifier obtained by inputting NPC values of 211 genes in Example 2

FIG. 4 is a Venn diagram of 23 genes for constructing a 0-(1+2) classifier and 15 genes for constructing a 1-2 classifier

FIG. 5 shows confusion matrices of a 15-gene SVM-RBF kernel three-class classifier obtained by inputting NPC values of 39 genes in Example 3

### DETAILED DESCRIPTION

The following further describes the present invention with examples, but the protection scope of the present invention is not limited thereto.

A person skilled in the art should understand that the examples of the present invention may be provided as a system or a computer program product. Therefore, the present invention may use a form of a hardware-only example, a software-only example, or an example with a combination of software and hardware. In addition, the present invention may use a form of a computer program product that is implemented on one or more computer-usable storage media that include computer-usable program code. The storage medium may be implemented by any type of volatile or non-volatile storage device or a combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic disk, or an optical disc. These computer program instructions may alternatively be stored in a computer-readable memory that can instruct a computer or another programmable data processing device to work in a specific manner, so that instructions stored in the computer-readable memory generate an artifact that includes an instruction apparatus. The instruction apparatus implements a function of early identification and severity prediction of placenta accreta spectrum.

### Example 1

PAS high-risk cohorts were established in a maternal and child health care hospital in city A (BGI sequencing platform), a maternal and child health care hospital in city B (Illumina sequencing platform), and a maternal and child health care hospital in city C (Life platform). NIPT data and clinical information were prospectively collected from pregnant women who underwent non-invasive DNA screening and possessed high-risk factors of PAS (at least one of history of uterine surgery such as cesarean section and myomectomy, history of hysteroscopic surgery or intrauterine procedures, and pregnancy achieved via assisted reproductive technology). Follow-up was conducted to 28 days postpartum.

Exclusion criteria include: (1) non-live birth outcomes such as stillbirth and neonatal death; (2) fetal chromosomal abnormalities or developmental malformations; and (3) multiple gestations. Pregnant women with occurrence of PAS and pregnant women without occurrence of PAS were matched based on maternal age, gestational age at NIPT, fetal sex, and distribution of high-risk factors.

In the high-risk pregnant women who underwent NIPT on the BGI sequencing platform, 70 cases of PAS pregnant women and 210 cases of non-PAS high-risk pregnant women were included in a training dataset, and 35 cases of PAS pregnant women and 95 cases of non-PAS high-risk pregnant women were included in an internal validation dataset, for differential analysis and model training. In addition, 51 cases of PAS pregnant women and 163 cases of non-PAS high-risk pregnant women who underwent NIPT PLUS detection (with a higher sequencing depth) on the BGI sequencing platform in the maternal and child health care hospital in city A, 54 cases of PAS pregnant women and 162 cases of non-PAS high-risk pregnant women enrolled in the maternal and child health care hospital in city B, and 55 cases of PAS pregnant women and 165 cases of non-PAS high-risk pregnant women enrolled in the maternal and child health care hospital in city C were included respectively in three independent external validation datasets (an NIPT PLUS dataset, an Illumina validation set, and a Life validation set).

NIPT data from the enrolled pregnant women was annotated with cfDNA promoter nucleosome coverage profiles and aligned to human reference genome hg19. PCR duplicates were removed. Original read coverages at pTSS regions were calculated. An NPC value of each gene was obtained and input to a classifier training system.

$${NPC}_{i} = \frac{{q}_{i} / {l}_{i}}{{\sum }_{j} ( {q}_{j} / {l}_{j} )} * {10}^{6} = \frac{{q}_{i}}{{\sum }_{j} {q}_{j}} * {10}^{6}$$

wherein, NPCᵢ represents a normalized pTSS coverage of a gene i, qᵢ represents an original read coverage at the pTSS region, lᵢ represents a transcript length (all being 2000), and Σⱼ(qⱼ/lⱼ) represents a sum of pTSS read coverages of all genes normalized based on the transcript length in one sample.

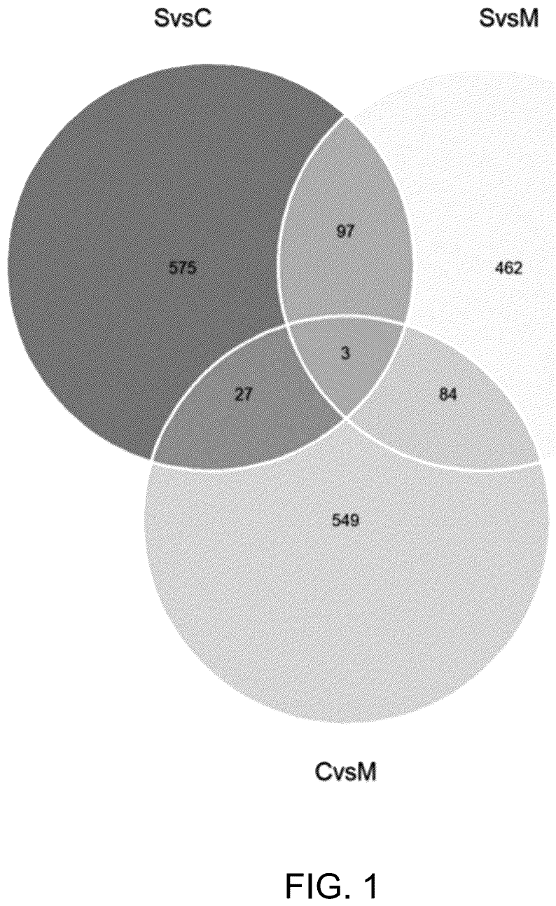

High-risk pregnant women in a severe PAS group and high-risk pregnant women without occurrence of PAS were matched at 1:1 and high-risk pregnant women in a mild PAS group and high-risk pregnant women without occurrence of PAS were matched at 1:1 in the training dataset by propensity scoring, and pairwise differential analyses were performed on the three matched groups. The impact factors in each two groups were subjected to three differential analysis methods: DESeq2, limma-voom, and a rank-sum test, to obtain impact factors with a p value<0.05 as the feature factors in the two groups. Intersection analysis was performed on three groups of feature factors obtained respectively through three times of screening, and feature factors obtained through all at least two times of screening were selected as optimal feature factors, which were 211 factors, and included in a next module. FIG. 1 is a Venn diagram of differential gene screening.

A discretization strategy was implemented to enhance universality and clinical utility of the classifier for different sequencing platforms. One group with an average NPC value of a gene significantly higher or lower than the other two groups in the three groups is used as a specificity group of the gene. The optimal cutoff value is defined as a NPC value with a maximum Youden index between the specificity group and the other two groups (that is, a maximum sum of sensitivity and specificity). A case in which a feature factor NPC is greater than a corresponding optimal cutoff value is set to 1; and a case in which a feature factor NPC is not greater than a corresponding optimal cutoff value is set to 0.

The selected 211 feature factors were input to an RFE process, and SVM-RBF kernel was selected for classifier training. PAS pregnant women set to 1 and non-PAS high-risk pregnant women set to 0 were input to the system for classifier training. To increase sensitivity of the classifier, the class weight parameter was set to {0:0.1, 1:0.3}. A disease risk assessment result of a to-be-predicted target was acquired, and k-fold cross-validation (k=10) was applied to enhance assessment robustness. An optimal feature factor combination is extracted, and an optimal classifier and an assessment result thereof are output.

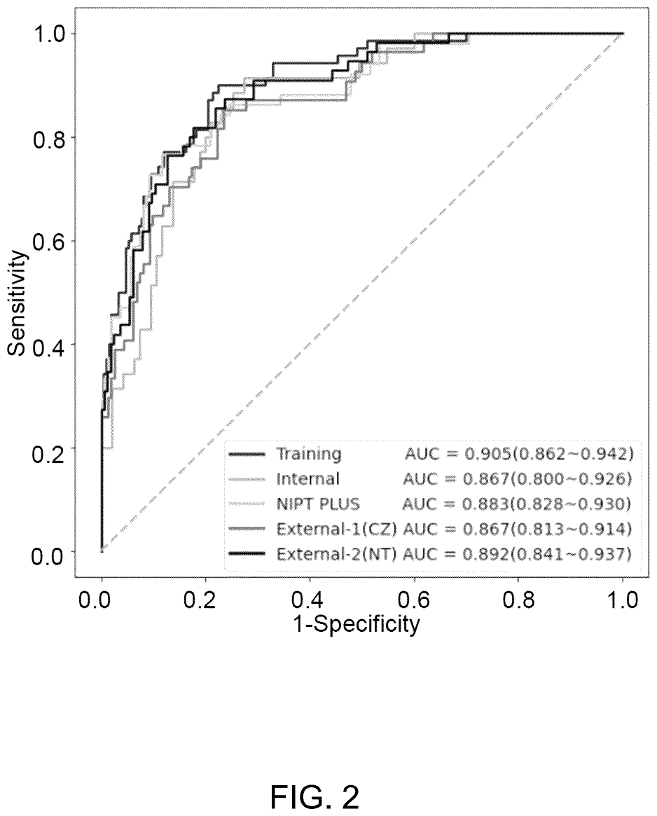

By using the SVM-RBF kernel training method, an output result is a 23-gene SVM-RBF kernel 0-(1+2) classifier. The AUC, accuracy, sensitivity, and specificity of this classifier were calculated in the training dataset, the internal validation dataset, and the external validation dataset, as shown in Table 1 and FIG. 2. The result indicates that the detection of expression levels of the 23 genes can accurately identify high-risk pregnant women with occurrence of PAS.

TABLE 1

| | 23-gene SVM-RBF kernel 0-(1 + 2) classifier | | | |
|---|---|---|---|---|
| | obtained by inputting NPC values of 211 genes | | | |
| Dataset | AUC | Accuracy | Sensitivity | Specificity |
| Training dataset | 0.905 (0.862-0.942) | 0.779 | 0.900 | 0.738 |
| Internal validation set | 0.867 (0.800-0.926) | 0.777 | 0.914 | 0.726 |
| PLUS validation set | 0.883 (0.828-0.930) | 0.752 | 0.863 | 0.718 |
| External validation set 1 | 0.867 (0.813-0.914) | 0.755 | 0.870 | 0.716 |
| External validation set 2 | 0.892 (0.841-0.937) | 0.759 | 0.909 | 0.709 |

### Example 2

Cohort establishment, sample collection, dataset division, data annotation, and feature factor screening are implemented by using the methods as described in Example 1. NPC values of 211 genes were input to a classifier training system.

The 211 feature factors were input to an RFE process, and SVM-RBF kernel was selected for classifier training. Pregnant women in a severe PAS group that were set to 2 and pregnant women in a mild PAS group that were set to 1 were input to the system for classifier training. To increase sensitivity of the classifier, the class weight parameter was set to {1:0.1, 2:0.3}. A disease risk assessment result of a to-be-predicted target was acquired, and k-fold cross-validation (k=10) was applied to enhance assessment robustness. Optimal feature factor combinations across different modeling methods were extracted, and a classifier and an assessment result thereof were output.

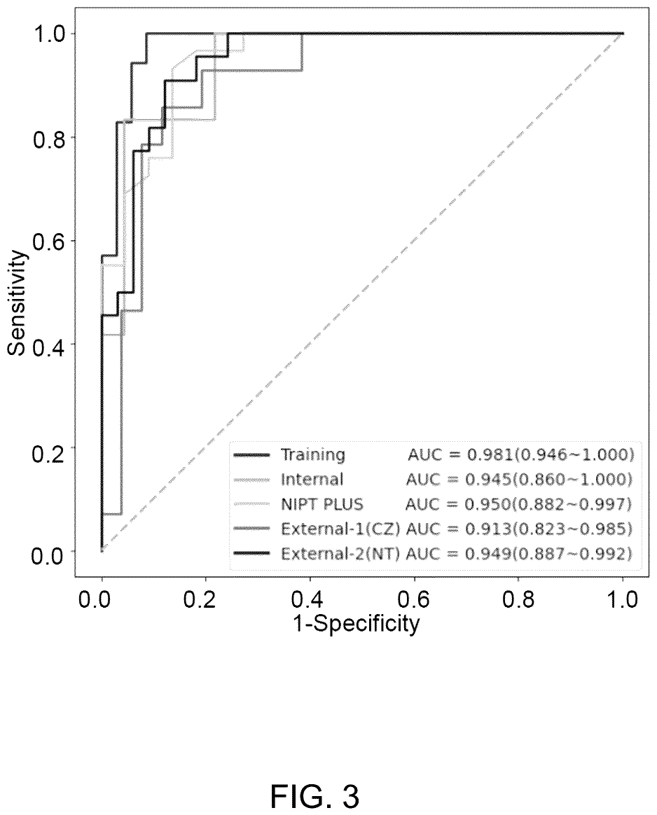

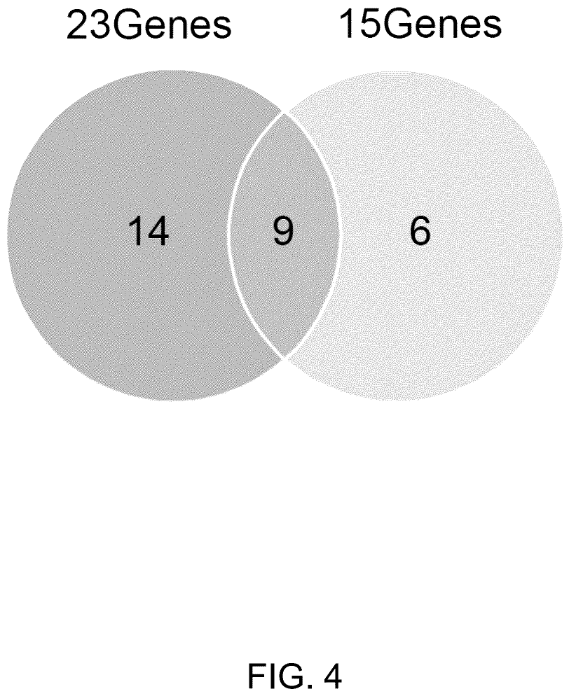

By using the SVM-RBF kernel training method, an output result is a 15-gene SVM-RBF kernel 1-2 classifier. The AUC, accuracy, sensitivity, and specificity of this classifier were calculated in the training dataset, the internal validation dataset, and the external validation dataset, as shown in Table 2 and FIG. 3. The overlap between 15 target genes of the 1-2 classifier and 23 target genes of the 0-(1+2) classifier is shown in FIG. 4. The result indicates that the detection of expression levels of the 15 genes can distinguish between high-risk pregnant women with severe PAS and high-risk pregnant women with mild PAS.

TABLE 2

| | 15-gene SVM-RBF kernel 1-2 classifier obtained | | | |
|---|---|---|---|---|
| | by inputting NPC values of 211 genes | | | |
| Dataset | AUC | Accuracy | Sensitivity | Specificity |
| Training dataset | 0.981 (0.946-1.000) | 0.929 | 0.914 | 0.943 |
| Internal validation set | 0.945 (0.860-1.000) | 0.829 | 0.917 | 0.783 |
| PLUS validation set | 0.950 (0.882-0.997) | 0.882 | 0.966 | 0.773 |
| External validation set 1 | 0.913 (0.823-0.985) | 0.870 | 0.857 | 0.885 |
| External validation set 2 | 0.949 (0.887-0.992) | 0.873 | 0.773 | 0.939 |

### Example 3

Cohort establishment, sample collection, dataset division, data annotation, and feature factor screening are implemented by using the methods as described in Example 1. NPC values of 23 genes in Example 1 and 15 genes in Example 2 (with discretization thresholds shown in Table 3) were input to an RFE process, and SVM-RBF kernel was selected for classifier training. Pregnant women with severe PAS that were set to 2, pregnant women with mild PAS that were set to 1, and non-PAS high-risk pregnant women set to 0 were input to the system for classifier training. To increase sensitivity of the classifier, the class weight parameter was set to {0:1, 1:3, 2:4}. A disease risk assessment result of a to-be-predicted target was acquired, and k-fold cross-validation (k=10) was applied to enhance assessment robustness. Optimal feature factor combinations across different modeling methods were extracted, and a classifier and an assessment result thereof were output.

TABLE 3

| | | Average NPC | Average NPC | Average NPC | Discretized | Optimal |
|---|---|---|---|---|---|---|
| | Specificity | value in the | value in the | value in the | single-gene | cutoff |
| Gene | group | severe group | mild group | control group | ROC value | value |
| CIGALTIC1 | Mild group | 34.842 | 45.022 | 35.840 | 0.702 | 45.162 |
| CEP192 | Mild group | 28.290 | 20.979 | 26.017 | 0.669 | 15.190 |
| KRT33A | Severe group | 17.059 | 24.190 | 22.853 | 0.645 | 16.599 |
| LINC02330 | Mild group | 39.788 | 45.925 | 41.469 | 0.582 | 41.476 |
| LOC644634 | Severe group | 22.487 | 29.255 | 28.945 | 0.609 | 18.252 |
| LOC728739 | Severe group | 45.882 | 37.757 | 39.038 | 0.592 | 44.508 |
| LPCAT3 | Mild group | 21.758 | 16.672 | 23.816 | 0.631 | 16.187 |
| MAPK10 | Control group | 42.479 | 44.672 | 36.852 | 0.586 | 42.586 |
| MIR12133 | Mild group | 42.319 | 33.618 | 43.368 | 0.634 | 43.341 |
| MIR1263 | Mild group | 40.387 | 33.779 | 40.240 | 0.630 | 42.838 |
| MIR3169 | Mild group | 42.924 | 36.998 | 44.280 | 0.616 | 35.299 |
| OR4N2 | Control group | 56.945 | 56.470 | 44.623 | 0.581 | 36.534 |
| OR7E2P | Severe group | 51.159 | 41.093 | 42.862 | 0.692 | 38.967 |
| RBFOX2 | Mild group | 42.651 | 51.957 | 42.848 | 0.616 | 44.923 |
| ZDHHC6 | Severe group | 39.497 | 49.032 | 46.539 | 0.636 | 34.135 |

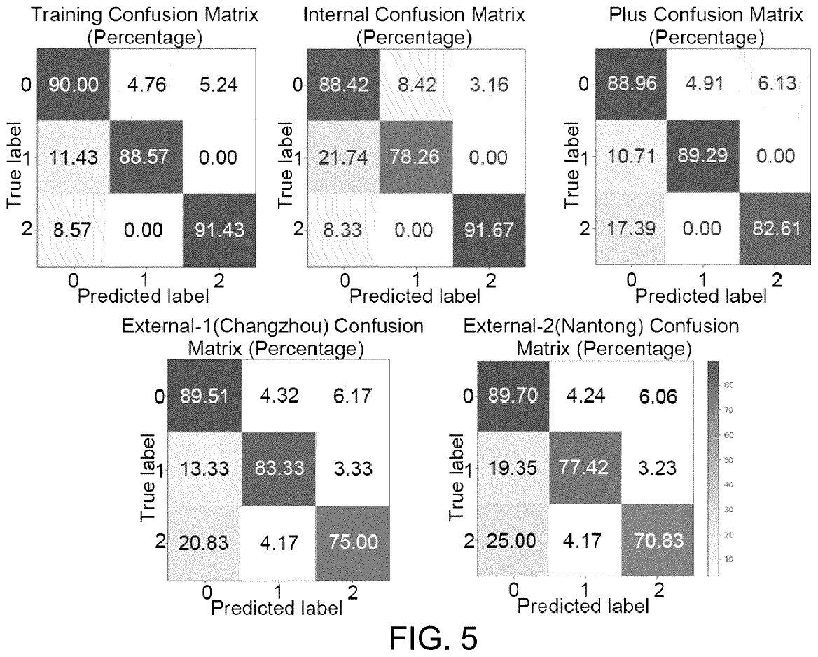

By using the SVM-RBF kernel training method, an optimal feature factor combination is extracted, a 15-gene SVM-RBF kernel three-class classifier is obtained, and an assessment result of the classifier are output, including average performance of the classifier and identification results of PAS, severe PAS, and mild PAS, as shown in Table 4 and FIG. 5. This result indicates that accurate early identification and risk prediction can be implemented on PAS by integrating expression levels of the 15 genes: MAPK10, MIR3169, MIR12133, ZDHHC6, LOC644634, LOC728739, KRT33A, C1GALT1C1, MIR1263, RBFOX2, CEP192, LINC02330, LPCAT3, OR7E2P, and OR4N2, and a detection method can be designed with high sensitivity and high specificity, holding promise for preparation of a diagnostic kit widely used in clinical diagnosis, advancing personalized medicine and precision treatment.

TABLE 4

| 15-gene | | | | | | | | | | | | | |
|---|---|---|---|---|---|---|---|---|---|---|---|---|---|
| SVM- | | | | | | | | | | | | | |
| RBF | | | | | | | | | | | | | |
| kernel | | | | | | | | | | | | | |
| three- | Average performance | | | | Occurrence of PAS | | | Severe PAS | | | Mild PAS | | |
| class | | Accur- | Sensit- | Speci- | | Sensit- | Speci- | | Sensit- | Speci- | | Sensit- | Speci- |
| classifier | AUC | acy | ivity | ficity | AUC | ivity | ficity | AUC | ivity | ficity | AUC | ivity | ficity |
| Training | 0.973 | 0.900 | 0.900 | 0.938 | 0.953 | 0.886 | 0.959 | 0.989 | 0.914 | 0.955 | 0.975 | 0.886 | 0.959 |
| dataset | (0.958- | | | | | | | | | | | | |
| | 0.986) | | | | | | | | | | | | |
| Internal | 0.937 | 0.869 | 0.861 | 0.909 | 0.934 | 0.783 | 0.925 | 0.945 | 0.917 | 0.975 | 0.956 | 0.783 | 0.925 |
| validation | (0.884- | | | | | | | | | | | | |
| set | 0.974) | | | | | | | | | | | | |
| PLUS | 0.946 | 0.883 | 0.870 | 0.922 | 0.908 | 0.890 | 0.863 | 0.956 | 0.826 | 0.948 | 0.971 | 0.893 | 0.957 |
| validation | (0.811- | | | | | | | | | | | | |
| set | 0.976) | | | | | | | | | | | | |
| External | 0.908 | 0.870 | 0.826 | 0.826 | 0.897 | 0.895 | 0.833 | 0.884 | 0.750 | 0.943 | 0.935 | 0.833 | 0.957 |
| validation | (0.857- | | | | | | | | | | | | |
| set 1 | 0.951) | | | | | | | | | | | | |
| External | 0.882 | 0.859 | 0.793 | 0.894 | 0.852 | 0.897 | 0.782 | 0.879 | 0.708 | 0.944 | 0.890 | 0.774 | 0.958 |
| validation | (0.826- | | | | | | | | | | | | |
| set 2 | 0.934) | | | | | | | | | | | | |

It is noted that the dataset division module, the impact factor extraction module, the feature selection module, the feature factor discretization module, and the model acquisition module may be implemented with the help of software and a necessary general-purpose hardware platform. For example, in an exemplary embodiment of the present invention, the classifier training system may also include an output apparatus, an input apparatus, a storage apparatus, and a control circuit. Instructions and functions executed by the dataset division module, the impact factor extraction module, the feature selection module, the feature factor discretization module, and the model acquisition module may run in the control circuit. For example, the control circuit is a central processing unit (CPU), another programmable general-purpose or special-purpose microprocessor, a digital signal processor (DSP), a programmable controller, an application-specific integrated circuit (ASIC), another similar element, or a combination thereof, and can run the dataset division module, the impact factor extraction module, the feature selection module, the feature factor discretization module, and the model acquisition module, to implement the function of constructing the classifier for early identification and severity prediction of placenta accreta spectrum. The classifier for early identification and severity prediction of placenta accreta spectrum provided in this application may be implemented with the help of software and a necessary general-purpose hardware platform. A computer software product is stored in a storage medium (such as ROM/RAM, a magnetic disk, or an optical disc), and includes several instructions for a terminal device (which may be a mobile phone, a computer, a server, a controlled terminal, or a network device) to perform the method in each example of this application.

The specific examples described herein are merely illustrative examples of the spirit of the present invention. A person skilled in the art to which the present invention pertains may make various modifications or additions or similar replacements to the specific examples, without departing from the spirit of the present invention or exceeding the scope defined by the appended claims.

1. A classifier training system for early identification and severity prediction of placenta accreta spectrum, comprising:
a dataset division module, configured to extract pre-collected medical data of placenta accreta spectrum (PAS) high-risk pregnant women, and divide the medical data into a discovery dataset, a training dataset, an internal validation dataset, and an external validation dataset;
an impact factor extraction module, configured to perform annotation on pre-collected non-invasive prenatal testing (NIPT) data of the PAS high-risk pregnant women with genome-wide cell-free DNA promoter nucleosome coverage profiles and perform feature extraction; calculate original read coverages at pTSS regions, wherein NIPT data is aligned to human reference genome hg19 by using a sequence alignment algorithm, PCR duplicates are removed, a region from −1000 bp to +1000 bp around a transcription start site (TSS) is determined as a promoter region pTSS, and the original read coverage at the pTSS region is calculated; and perform TPM-like normalization on the original read coverages at the pTSS regions to obtain normalized pTSS coverages NPC, wherein NPC corresponding to each gene is used as an impact factor;
a feature selection module, configured to screen impact factors of high-risk pregnant women with severe PAS, high-risk pregnant women with mild PAS, and high-risk pregnant women without occurrence of PAS in the discovery dataset, to determine feature factors;
a feature factor discretization module, configured to determine an optimal cutoff value of each feature factor, and perform discretization on the feature factors based on the optimal cutoff value; and
a model acquisition module, configured to input the determined feature factors to a recursive feature elimination (RFE) process, screen, by using a plurality of machine learning algorithms, for characteristic gene sets respectively representing occurrence of PAS and types of PAS, combine the two gene sets and then input the combined gene set to an RFE process, gradually construct a PAS three-class classifier; acquire a comprehensive disease risk assessment result of a to-be-predicted target, apply k-fold cross-validation to enhance assessment robustness; and extract an optimal feature factor combination, and output a classifier and an assessment result thereof.

2. The system according to claim 1, wherein in the dataset division module, pregnant women with severe PAS, pregnant women with mild PAS, and pregnant women without occurrence of PAS are matched based on maternal age, gestational age at NIPT, fetal sex, and distribution of high-risk factors, samples from a primary center are randomly divided into the training dataset and the internal validation dataset, samples from each sub-center are used as the external validation dataset, and matching is performed on the training dataset at 1:1 based on the gestational age at NIPT and the fetal sex to obtain the discovery dataset.

3. The system according to claim 1, wherein in the impact factor extraction module, the NIPT data is aligned to the human reference genome hg19 by using the sequence alignment algorithm, the PCR duplicates are removed, the region from −1000 bp to +1000 bp around the transcription start site (TSS) is determined as the promoter region pTSS, and the original read coverage at the pTSS region is calculated.

4. The system according to claim 3, wherein in the impact factor extraction module, TPM-like normalization is performed on the original read coverage at the pTSS region, wherein NPC is obtained through the following formula:
$${NPC}_{i} = \frac{{q}_{i} / {l}_{i}}{{\sum }_{j} ( {q}_{j} / {l}_{j} )} * {10}^{6} = \frac{{q}_{i}}{{\sum }_{j} {q}_{j}} * {10}^{6}$$
wherein NPCᵢ represents a normalized pTSS coverage of a gene i, qᵢ represents an original read coverage at the pTSS region, lᵢ represents a transcript length, and Σⱼ(qⱼ/lⱼ) represents a sum of pTSS read coverages of all genes normalized based on the transcript length in one sample.

5. The system according to claim 1, wherein in the feature selection module, high-risk pregnant women in a severe PAS group and high-risk pregnant women without occurrence of PAS are matched at 1:1 and high-risk pregnant women in a mild PAS group and high-risk pregnant women without occurrence of PAS are matched at 1:1 in the training dataset by propensity scoring, and pairwise differential analyses are performed on the three matched groups;
the impact factors in each two groups are subjected to three differential analysis methods: DESeq2, limma-voom, and a rank-sum test, to obtain impact factors with a p value of less than 0.05 as the feature factors in the two groups; and
intersection analysis is performed on three groups of feature factors obtained respectively through three times of screening, and feature factors obtained through all three times of screening or through all at least two times of screening are selected as optimal feature factors.

6. The system according to claim 1, wherein in the feature factor discretization module, to enhance universality and clinical utility of the classifier for different sequencing platforms, one group with an average NPC value of a gene significantly higher or lower than the other two groups in the three groups is used as a specificity group of the gene; the optimal cutoff value is defined as a NPC value with a maximum Youden index between the specificity group and the other two groups; a case in which a feature factor NPC is greater than a corresponding optimal cutoff value is set to 1; and a case in which a feature factor NPC is not greater than a corresponding optimal cutoff value is set to 0.

7. The system according to claim 1, wherein in the model acquisition module,
the optimal feature factor combination comprises the following target genes: MAPK10, MIR3169, MIR12133, ZDHHC6, LOC644634, LOC728739, KRT33A, C1GALT1C1, MIR1263, RBFOX2, CEP192, LINC02330, LPCAT3, OR7E2P, and OR4N2; and
support vector machine (SVM)-RBF kernel is selected for the classifier.

8. A classifier for early identification of a placenta accreta spectrum risk in high-risk pregnant women, obtained through the training system according claim 1.

9. The system according to claim 1, use of a reagent for detecting expression of genes MAPK10, MIR3169, MIR12133, ZDHHC6, LOC644634, LOC728739, KRT33A, C1GALT1C1, MIR1263, RBFOX2, CEP192, LINC02330, LPCAT3, OR7E2P, and OR4N2 in preparation of a diagnostic kit for early identification and severity prediction of placenta accreta spectrum.

10. A classifier for early identification of a placenta accreta spectrum risk in high-risk pregnant women, obtained through the training system according claim 2.

11. A classifier for early identification of a placenta accreta spectrum risk in high-risk pregnant women, obtained through the training system according claim 3.

12. A classifier for early identification of a placenta accreta spectrum risk in high-risk pregnant women, obtained through the training system according claim 4.

13. A classifier for early identification of a placenta accreta spectrum risk in high-risk pregnant women, obtained through the training system according claim 5.

14. A classifier for early identification of a placenta accreta spectrum risk in high-risk pregnant women, obtained through the training system according claim 6.

15. A classifier for early identification of a placenta accreta spectrum risk in high-risk pregnant women, obtained through the training system according claim 7.
